(19) United States
(12) Patent Application Publication
(10) Pub. No.: US 2025/0292882 A1
(43) Pub. Date: Sep. 18, 2025

(54) MEDICAL INFORMATION MANAGEMENT APPARATUS, MEDICAL INFORMATION MANAGEMENT SYSTEM, MEDICAL INFORMATION MANAGEMENT METHOD, AND STORAGE MEDIUM

(71) Applicant: Konica Minolta, Inc., Tokyo (JP)

(72) Inventor: Hiroshi NAKAMORI, Tokyo (JP)

(21) Appl. No.: 19/078,672

(22) Filed: Mar. 13, 2025

(30) Foreign Application Priority Data
Mar. 18, 2024 (JP) 2024-041871

(51) Int. Cl. G16H 10/60; G16H 40/20

(52) U.S. Cl. CPC G16H 10/60; G16H 40/20

(57) ABSTRACT

A medical information management apparatus includes a hardware processor. The hardware processor requests that medical data stored in a first storage apparatus having a predetermined access speed be stored in a second storage device having a lower access speed than the first storage device. The hardware processor instructs suppressing deletion of the medical data stored in the first storage device. The hardware processor requests control to suppress the deletion of the medical data for which the suppressing of the deletion is instructed.

### BACKGROUND OF THE INVENTION

### Technical Field

The present invention relates to a medical information management apparatus, a medical information management system, a medical information management method, and a storage medium.

### Description of Related Art

In recent years, a cloud storage has been used, which can substitute for an external storage device such as a tape or a DVD whose maintenance and management are troublesome, and can keep initial investment low. As the cloud storage, a high-speed storage that can be accessed at high speed, a low-speed storage that is accessed at a lower speed than the high-speed storage, and the like are provided. For example, data with a high reference frequency is stored in the high-speed storage, and data with a low reference frequency is moved from the high-speed storage to the low-speed storage. When the medical image moved to the low-speed storage is referred to, it takes time to take out the medical image.

Japanese Unexamined Patent Publication No. H10-143402 describes a medical image storage communication system including a first storage device having a low access speed and a second storage device having a high access speed. In the medical image storage communication system, medical image information whose possibility of reference has decreased is deleted from the second storage device.

However, depending on the patient, there are cases in which the healing state is observed for a long period of time, such as performing follow-up observation once every half a year. In such a case, there is a demand that a medical image which has a low possibility of being referred to but needs to be continuously referred to be originally left in the high-speed storage. However, the conventional technique has a problem in that medical images having a low possibility of being referred to are uniformly deleted from the high-speed storage.

### SUMMARY OF THE INVENTION

Therefore, in order to solve the above-described problem, an object of the present invention is to provide a medical information management apparatus, a medical information management system, a medical information management method, and a storage medium including a program capable of suppressing by a user's instruction deletion of any medical image even in a case in which the medical image is automatically deleted from a storage device capable of high-speed access under a predetermined condition.

A medical information management apparatus according to the present invention includes
a hardware processor,
wherein,
the hardware processor requests that medical data stored in a first storage apparatus having a predetermined access speed be stored in a second storage device having a lower access speed than the first storage device,
the hardware processor instructs suppressing deletion of the medical data stored in the first storage device, and
the hardware processor requests control to suppress the deletion of the medical data for which the suppressing of the deletion is instructed.

A medical information management system according to the present invention includes
a hardware processor,
wherein,
the hardware processor requests that medical data stored in a first storage apparatus having a predetermined access speed be stored in a second storage device having a lower access speed than the first storage device,
the hardware processor instructs suppressing deletion of the medical data stored in the first storage device, and
the hardware processor requests control to suppress the deletion of the medical data for which the suppressing of the deletion is instructed.

A medical information management method according to the present invention includes the steps of:
requesting that medical data stored in a first storage apparatus having a predetermined access speed be stored in a second storage device having a lower access speed than the first storage device;
instructing suppressing deletion of the medical data stored in the first storage device; and
requesting control to suppress the deletion of the medical data for which the suppressing of the deletion is instructed.

A non-transitory computer-readable storage medium comprising a program for causing a computer to perform:
requesting that medical data stored in a first storage apparatus having a predetermined access speed be stored in a second storage device having a lower access speed than the first storage device;
instructing suppressing deletion of the medical data stored in the first storage device; and
requesting control to suppress the deletion of the medical data for which the suppressing of the deletion is instructed.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram showing an example of a schematic configuration of a medical information management system according to a first embodiment;

FIG. 2 is a diagram illustrating an example of a configuration of an examination list screen displayed on a display part of the medical information management apparatus according to the first embodiment;

FIG. 3 is a diagram illustrating an example of the configuration of a table according to the first embodiment;

FIG. 4 is a flowchart illustrating an example of an operation of the medical information management system according to the first embodiment in a case in which control such as movement of medical data stored in the high-speed storage to the low-speed storage and suppression of the movement is executed;

FIG. 5 is a diagram illustrating an example of a schematic configuration of the medical information management system according to a first modification example of the first embodiment;

FIG. 6 is a diagram illustrating an example of a schematic configuration of the medical information management system according to a second modification example of the first embodiment;

FIG. 7 is a diagram illustrating an example of a schematic configuration of the medical information management system according to a third modification example of the first embodiment;

FIG. 8 is a diagram illustrating an example of a schematic configuration of the medical information management system according to the second embodiment;

FIG. 9 is a flowchart illustrating an example of an operation of the medical information management system according to the second embodiment in a case in which control such as copying of medical data stored in the high-speed storage to the low-speed storage and suppressing of the copying is executed;

FIG. 10 is a diagram illustrating an example of a schematic configuration of the medical information management system according to a first modification example of the second embodiment;

FIG. 11 is a diagram illustrating an example of a schematic configuration of the medical information management system according to a second modification example of the second embodiment; and

FIG. 12 is a diagram illustrating an example of a schematic configuration of the medical information management system according to a third modification example of the second embodiment.

### DETAILED DESCRIPTION

Hereinafter, one or more embodiments of the present invention will be described with reference to the drawings. However, the scope of the invention is not limited to the disclosed embodiments.

A medical information management apparatus, a medical information management system, a medical information management method, and a storage medium including a program according to preferred embodiments of the present disclosure will be described in detail below with reference to the accompanying drawings.

### 1. First Embodiment

### [Schematic Configuration Example of Medical Information Management System 1A]

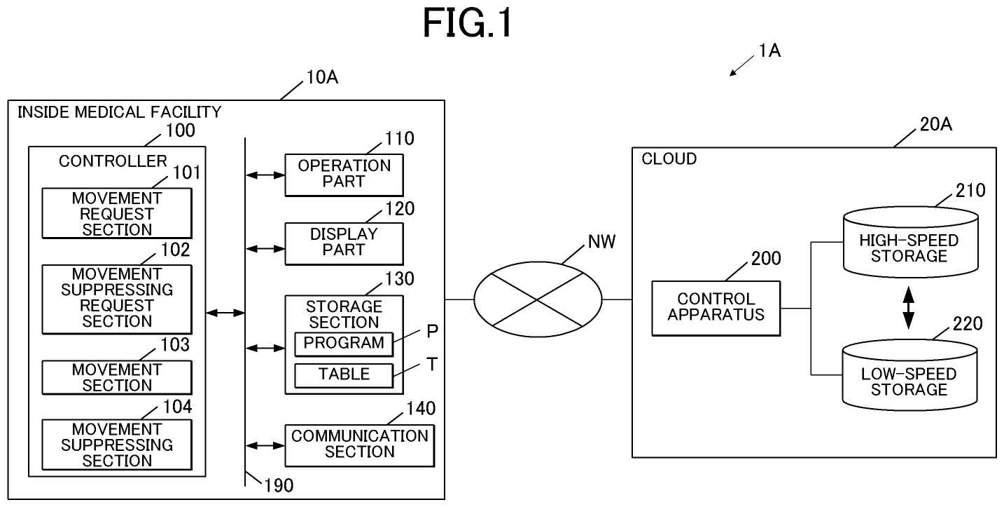

FIG. 1 is a diagram illustrating an example of a schematic configuration of a medical information management system 1A according to a first embodiment. The medical information management system 1A includes a medical information management apparatus 10A provided in a medical facility and a data storage system 20A provided on a cloud. The medical facilities include, for example, hospitals, clinics, medical checkup centers, and other medical institutions. The medical information management apparatus 10A and the data storage system 20A are communicably connected to each other via a network NW. Examples of the network NW include the Internet, a wide area network (WAN), and a telephone communication network. WAN is an abbreviation for Wide Area Network.

The medical information management apparatus 10A is, for example, a PACS and manages and stores therein medical data imaged by respective modalities. PACS is an abbreviation for Picture Archiving and Communication System. Examples of the modality include an X-ray imaging apparatus, a CT apparatus, an MR apparatus, and an ultrasonic diagnostic apparatus. CT is an abbreviation for Computed Tomography. MR is an abbreviation for Magnetic Resonance. The medical data is various images acquired by the X-ray imaging apparatus, the CT apparatus, the MR apparatus, the ultrasound diagnostic apparatus, and the like, and includes a still image, a moving image, a stereoscopic image, and the like.

The data storage system 20A manages and stores therein the medical data transmitted from the medical information management apparatus 10A via the network NW. The data storage system 20A includes a high-speed storage 210 and a low-speed storage 220. The high-speed storage 210 is a storage device capable of high-speed access, and stores relatively new medical data, medical data with a high browsing frequency, and the like. The low-speed storage 220 is a storage device whose access speed is lower than that of the high-speed storage 210, and stores relatively old medical data, infrequently browsed medical data, and the like. The data storage system 20A reads medical data for which a reference request has been made from a viewer, a client terminal, or the like provided inside the medical facility or outside the medical facility, and transmits the read medical data to the viewer or the like as a request source. Note that the high-speed storage 210 corresponds to a first storage device, and the low-speed storage 220 corresponds to a second storage device.

### [Schematic Configuration Example of Medical Information Management Apparatus 10A]

Next, the medical information management apparatus 10A will be described in detail. The medical information management apparatus 10A includes a controller 100 (hardware processor), an operation part 110, a display part 120, a storage section 130, and a communication section 140. The controller 100, the operation part 110, the display part 120, the storage section 130, and the communication section 140 are connected to each other via wiring 190 such as a bus.

The controller 100 includes, for example, a processor such as a CPU that performs calculation and control, a memory, and the like. CPU is an abbreviation for Central Processing Unit. The controller 100 executes, for example, a program stored in a memory such as a RAM, the storage section 130, or the like to realize processing such as movement of medical data stored in the high-speed storage 210 to the low-speed storage 220 and suppression of the movement.

Note that the controller 100 may include an electronic circuit such as an ASIC and an FPGA. ASIC is an abbreviation of Application Specific Integrated Circuit. FPGA is an abbreviation for Field Programmable Gate Array.

According to the present embodiment, the controller 100 functions as a movement request section 101, a movement suppressing request section 102, a movement section 103, and a movement suppressing section 104. The processor of the controller 100 executes a program P stored in the storage section 130 or the like, thereby implementing each function and process of a storage request step by the movement request section 101, a deletion suppression request step by the movement suppressing request section 102, and the like.

The movement request section 101 makes a request to move and store the medical data stored in the high-speed storage 210 to the low-speed storage 220. The movement refers to storing the medical data stored in the high-speed storage 210 into the low-speed storage 220 and deleting the medical data stored in the high-speed storage 210.

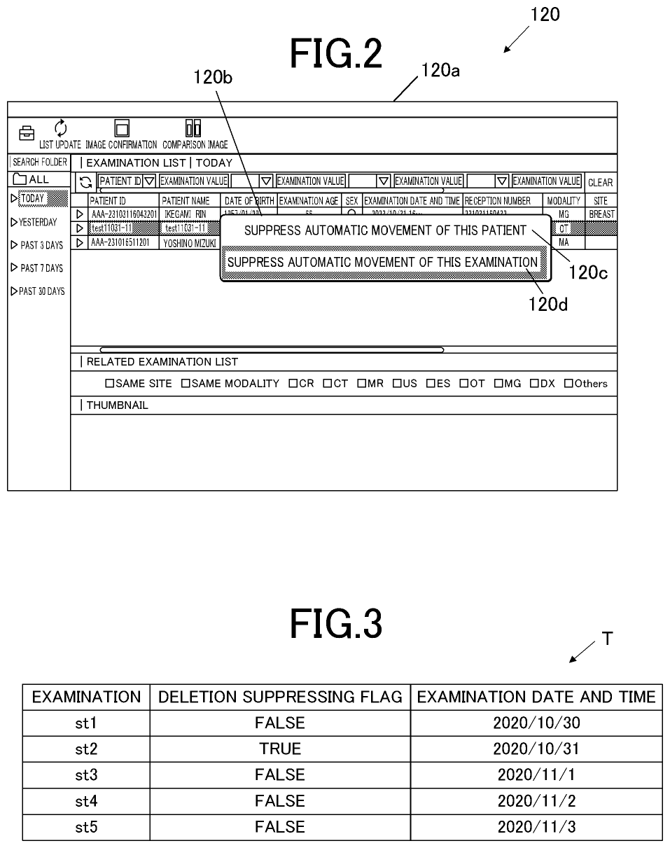

The movement suppressing request section 102 requests control to suppress deletion of medical data for which suppressing of deletion has been instructed by an instruction section (instructor) such as an examination list screen 120a (see FIG. 2). The request to suppress deletion of the medical data is a request to suppress movement of the medical data. The suppressing of the movement can be set on an examination basis or a patient basis by changing a deletion suppressing flag to be enabled by an instruction section such as the examination list screen 120a. The movement suppressing request section 102 may determine whether the deletion suppressing flag of the patient is valid. The movement suppressing request section 102 requests, among the medical data stored in the high-speed storage 210, control for suppressing the movement of medical data that is determined to be deleted on the basis of a predetermined movement condition. The movement suppressing request section 102 can preferentially suppress deletion of the medical data that is automatically deleted based on the movement condition.

The movement section 103 performs control to move and store the medical data stored in the high-speed storage 210 to the low-speed storage 220, based on the movement request of the movement request section 101. In this case, the movement section 103 performs control to automatically move based on a preset movement condition. The movement suppressing section 104 performs control to prevent the movement of the medical data stored in the high-speed storage 210 to the low-speed storage 220, based on the request of the movement suppressing request section 102.

The operation part 110 includes, for example, a mouse, a keyboard, a switch, and a button. The operation part 110 may be, for example, a touch screen integrally combined with a display. The operation part 110 accepts instructions corresponding to various types of input operation from the user, converts the accepted instruction into an operation signal, and outputs the operation signal to the controller 100. Specifically, the operation part 110 accepts an instruction to suppress the movement of the medical data from the high-speed storage 210 to the low-speed storage 220, and the like. That is, the operation part 110 accepts by one instruction to suppress deletion of all medical data of a predetermined patient or medical data of a predetermined examination. Note that the operation part 110 functions as an instruction section and an instruction step.

The display part 120 is, for example, a display such as a liquid crystal display or an organic EL display. EL is an abbreviation for Electro Luminescence. The display part 120 displays the medical image acquired from the modality, a GUI for accepting various input operations from the user, and the like. GUI is an abbreviation for Graphical User Interface. Specifically, the display part 120 displays the examination list screen 120a on which it is possible to designate the suppressing of the movement of the medical data from the high-speed storage 210 to the low-speed storage 220. That is, the display part 120 displays the examination list screen 120a for selecting to suppress deletion of the one patient or the medical data of the one patient according to the one instruction. Note that the operation part 110 functions as an instruction section and an instruction step.

The storage section 130 includes, for example, any storage module such as an HDD, an SSD, a ROM, and a RAM. HDD is an abbreviation of Hard Disk Drive. SSD is an abbreviation of Solid State Drive. ROM is an abbreviation of Read Only Memory. The storage section 130 stores, for example, a system program, an application program, and various types of data. Specifically, the storage section 130 stores the program P for executing processing such as movement of the medical data stored in the high-speed storage 210 to the low-speed storage 220 and suppression of the movement. The storage section 130 stores a table T including a deletion suppressing flag indicating whether to move the medical data stored in the high-speed storage 210 to the low-speed storage 220 or to prevent the movement.

The communication section 140 includes, for example, a communication module including an NIC, a receiver, and a transmitter. NIC is an abbreviation for Network Interface Card. The communication section 140 communicates various kinds of information and data with an external data storage system 20A or the like provided on a cloud via the network NW.

In FIG. 1, the medical information management apparatus 10A includes the operation part 110 and the display part 120, but the medical information management apparatus 10A may not include the operation part 110 and the display part 120. For example, in each user terminal installed in the medical facility or the like, each doctor may display the examination list screen 120a or the like on a browser and input an instruction or the like to suppress deletion of medical data.

### [Schematic Configuration Example of Data Storage System 20A]

As shown in FIG. 1, the data storage system 20A includes a control apparatus 200 (hardware processor) in addition to the high-speed storage 210 and the low-speed storage 220 described above. The control apparatus 200 includes, for example, a processor such as a CPU that performs calculation and control, a memory, and the like. The control apparatus 200 executes control such as movement of medical data stored in the high-speed storage 210 to the low-speed storage 220 and suppressing of the movement based on each instruction from the medical information management apparatus 10A or the like.

The high-speed storage 210 and the low-speed storage 220 function as external storage devices or the like for backing up medical data in the medical information management apparatus 10A. The high-speed storage 210 is, for example, a solid state drive (SSD), and stores medical data transmitted from the medical information management apparatus 10A. Based on the movement instruction from the medical information management apparatus 10A, the high-speed storage 210 moves the medical data corresponding to the movement instruction to the low-speed storage 220. The low-speed storage 220 is, for example, a hard disk drive (HDD) or a recording medium, and stores the medical data moved from the high-speed storage 210.

[Example of Configuration of Examination List Screen 120a]

Next, a description will be given of the examination list screen 120a for setting whether or not to suppress the movement of the medical data from the high-speed storage 210 to the low-speed storage 220. FIG. 2 is a diagram illustrating an example of a configuration of an examination list screen 120a displayed on a display part 120 of the medical information management apparatus 10A according to the first embodiment. In the examination list screen 120a, a list of examination information performed on each patient is displayed. The examination information includes, for example, a patient ID, a patient name, a date of birth, an examination age, sex, and an examination date and time.

When any examination information is selected by the user in the list of the examination list screen 120a, a pop-up screen 120b is displayed. In the pop-up screen 120b, first selection information 120c “suppress automatic movement of this patient” and second selection information 120d “suppress automatic movement of this examination” are displayed. The first selection information 120c is a selection item for suppressing, on a patient-by-patient basis, the movement of the medical data stored in the high-speed storage 210 to the low-speed storage 220. In a case in which the first selection information 120c is selected, a deletion suppressing flag for suppressing the movement of all medical data of the selected patient from the high-speed storage 210 to the low-speed storage 220 is set. The second selection information 120d is a selection item for suppressing, on an examination basis, the movement of medical data stored in the high-speed storage 210 to the low-speed storage 220. When the second selection information 120d is selected, the deletion suppressing flag for suppressing the movement of the medical data of the selected examination is set.

### [Example of Configuration of Table T]

Next, an example of a configuration of a table T stored in the storage section 130 according to the present embodiment will be described. FIG. 3 is a diagram illustrating an example of the configuration of the table T according to the first embodiment. In the table T, examination information, deletion suppressing flag information, and examination date and time information are stored in association with each other. The examination information includes, for example, at least one or more information among a patient ID, a patient name, a receipt number, and the like. The deletion suppressing flag information includes information of either “true” or “false”. The flag “true” is a flag indicating movement of the medical data stored in the high-speed storage 210 to the low-speed storage 220 is suppressed. The flag “false” is a flag indicating movement of the medical data stored in the high-speed storage 210 to the low-speed storage 220. For example, when the user selects the suppression of the movement of the specific examination or the like of the patient on the examination list screen 120a, the deletion suppressing flag information of the examination or the like of the patient selected on the examination list screen 120a is set to “true”. Note that in the table T, the default of the deletion suppressing flag information is set to “false”.

### [Operation Example of Medical Information Management System 1A]

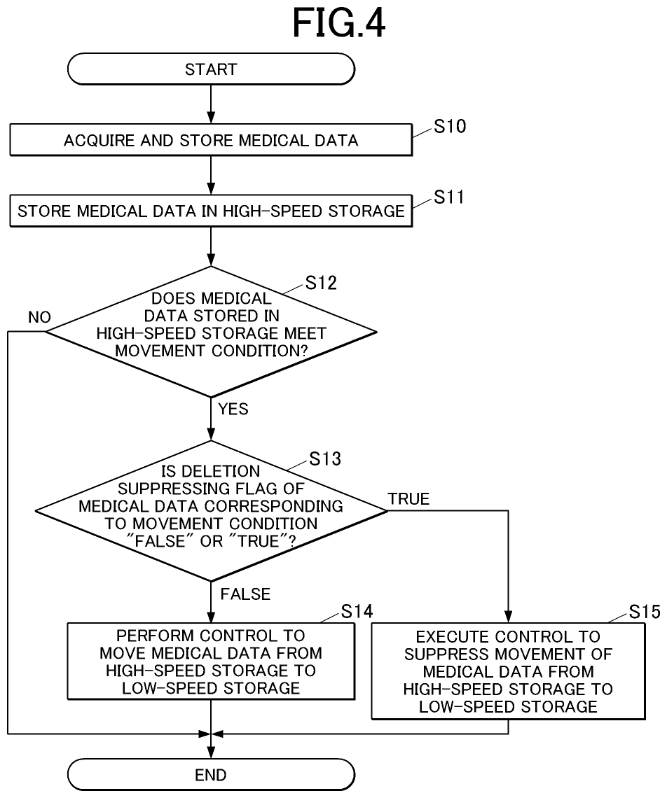

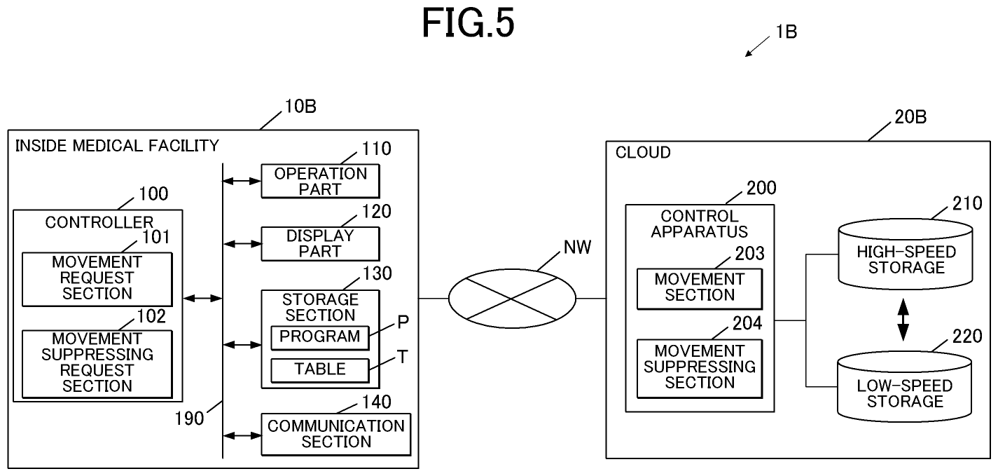

Next, a flow of control such as movement of the medical data stored in the high-speed storage 210 according to the present embodiment to the low-speed storage 220 and suppression of the movement will be described. FIG. 4 is a flowchart illustrating an example of an operation of the medical information management system 1A according to the present embodiment in a case in which control such as movement of medical data stored in the high-speed storage 210 to the low-speed storage 220 and suppression of the movement is executed.

The controller 100 of the medical information management apparatus 10A acquires the medical data from the modality, and stores the acquired medical data in the storage section 130 (step S10).

After storing the medical data acquired from the modality in the storage section 130, the controller 100 stores the medical data in the high-speed storage 210 of the data storage system 20A (step S11). Specifically, the controller 100 transmits the acquired medical data to the high-speed storage 210 via the network NW. The high-speed storage 210 stores therein the medical data transmitted from the medical information management apparatus 10A, on the basis of an instruction of the controller 100 of the medical information management apparatus 10A. Note that the medical data on the side of the medical information management apparatus 10A can be deleted from the storage section 130 after a lapse of a fixed period of time such as five years.

The controller 100 determines whether the medical data stored in the high-speed storage 210 satisfies a movement condition set in advance (step S12). The preset movement condition is, for example, a condition that a certain storage period has elapsed since the medical data was stored in the high-speed storage 210, a condition that the browsing frequency of the medical data stored in the high-speed storage 210 is less than a predetermined number of times, or the like. In a case where the controller 100 determines that the medical data stored in the high-speed storage 210 satisfies the movement condition, the process proceeds to step S13. On the other hand, when determining that the medical data stored in the high-speed storage 210 does not correspond to the movement condition, the controller 100 ends the series of processing without moving the medical data from the high-speed storage 210.

The controller 100 determines whether the deletion suppressing flag is set to “false” or “true” for the medical data corresponding to the movement condition (step S13). Specifically, the controller 100 refers to the table T and determines whether the deletion suppressing flag is set to “false” or the deletion suppressing flag is set to “true” for the medical data corresponding to the movement condition. If the controller 100 determines that the deletion suppressing flag for the medical data corresponding to the movement condition is set to “false”, the controller 100 proceeds to step S14. That is, this is a case where the medical data corresponds to the movement condition and the deletion suppressing flag is set to “false”.

The controller 100 sends, to the data storage system 20A, a movement instruction based on the request to move the medical data corresponding to the movement condition from the high-speed storage 210 to the low-speed storage 220. That is, the controller 100 executes control to move the medical data corresponding to the above movement condition from the high-speed storage 210 to the low-speed storage 220 (step S14). In this case, the controller 100 functions as the movement request section 101 and the movement section 103.

Upon receiving the movement instruction from the medical information management apparatus 10A, the high-speed storage 210 of the data storage system 20A moves the medical data corresponding to the movement condition to the low-speed storage 220. At this time, the medical data stored in the high-speed storage 210 is deleted from the high-speed storage 210 along with the movement to the low-speed storage 220.

On the other hand, if the controller 100 determines in step S13 that the deletion suppressing flag for the medical data corresponding to the movement condition is set to “true”, the controller 100 proceeds to step S15. That is, this is a case in which the medical data corresponds to the movement condition and the deletion suppressing flag is set to “true”.

The controller 100 executes control to suppress the movement of the medical data corresponding to the movement condition from the high-speed storage 210 to the low-speed storage 220 (step S15). In this case, the controller 100 functions as a movement suppressing request section 102 and a movement suppressing section 104. Thus, even the medical data corresponding to the movement condition is not moved from the high-speed storage 210 to the low-speed storage 220. Therefore, even if the medical data is determined to be deleted due to the movement, the medical data for which the deletion suppressing flag is set to “true” is not deleted from the high-speed storage 210.

According to the first embodiment, by enabling the deletion suppressing flag of one patient or the like on the examination list screen 120a or the like, it is possible to designate in advance the medical data for which the automatic movement of the medical data corresponding to the movement condition from the high-speed storage 210 to the low-speed storage 220 is suppressed. Thus, even when the medical data corresponds to the movement condition, the medical data can be left in the high-speed storage 210 by enabling the deletion suppressing flag, and an undesired movement to the low-speed storage 220 can be suppressed. As a result, for example, even medical data that is infrequently referred to can be referred to from the high-speed storage 210, thus reducing the standby time until it can be referred to.

Furthermore, according to the first embodiment, medical data to be suppressed from being automatically moved from the high-speed storage 210 to the low-speed storage 220 can be specified on the examination list screen 120a on a patient-by-patient basis and an examination-by-examination basis. Thus, the medical data to be suppressed from being moved from the high-speed storage 210 to the low-speed storage 220 can be efficiently specified according to the examination content of each patient.

### 1-1. First Modification Example of First Embodiment

In the first modification example, the data storage system 20A provided on the cloud executes the movement of the medical data stored in the high-speed storage 210 to the low-speed storage 220 and the suppressing of the movement. Note that in the first modification example, common constituent elements to the above-described first embodiment are denoted by the same reference signs, and redundant description is omitted.

### [Configuration Example of Medical Information Management System 1B]

FIG. 5 is a diagram illustrating an example of a schematic configuration of the medical information management system 1B according to a first modification example of the first embodiment. The medical information management system 1B includes a medical information management apparatus 10B provided in the medical facility and a data storage system 20B provided on a cloud.

The medical information management apparatus 10B includes a controller 100, an operation part 110, a display part 120, a storage section 130, and a communication section 140. The controller 100 functions as a movement request section 101 and a movement suppressing request section 102. The processor of the controller 100 executes a program P stored in the storage section 130 or the like to implement each function of the movement request section 101 or the like. Note that although the medical information management apparatus 10B is configured to include the operation part 110 and the display part 120 in FIG. 5, the medical information management apparatus 10B may be configured not to include the operation part 110 and the display part 120. For example, in each user terminal installed in the medical facility or the like, each doctor may display the examination list screen 120a or the like on a browser and input an instruction or the like to suppress deletion of medical data.

The data storage system 20B includes a control apparatus 200, a high-speed storage 210, and a low-speed storage 220. The control apparatus 200 functions as a movement section 203 and a movement suppressing section 204. The processor of the control apparatus 200 executes a program stored in a memory or the like (not illustrated) to realize each function of the movement section 203 and the like.

### [Operation Example of Medical Information Management System 1B]

Next, a description will be given of a flow in a case in which processing such as movement of the medical data stored in the high-speed storage 210 to the low-speed storage 220 and suppression of the movement is executed according to the first modification example.

The controller 100 of the medical information management apparatus 10B acquires the medical data from the modality, and stores the acquired medical data in the storage section 130. After storing the medical data acquired from the modality in the storage section 130, the controller 100 transmits the medical data to the high-speed storage 210 of the data storage system 20B via the network NW. The high-speed storage 210 stores the medical data transmitted from the medical information management apparatus 10B.

The controller 100 determines whether or not the medical data stored in the high-speed storage 210 corresponds to a movement condition set in advance. When the medical data stored in the high-speed storage 210 satisfies the movement condition, the controller 100 determines whether the deletion suppressing flag of the medical data is set to “false” or “true”. When the deletion suppressing flag is “false”, the controller 100 transmits, to the data storage system 20B, a movement request for moving the medical data corresponding to the movement condition from the high-speed storage 210 to the low-speed storage 220. In this case, the controller 100 functions as the movement request section 101.

Upon receiving the movement request from the medical information management apparatus 10B, the control apparatus 200 of the data storage system 20B outputs, to the high-speed storage 210, a movement instruction to move the medical data corresponding to the movement condition from the high-speed storage 210 to the low-speed storage 220. The high-speed storage 210 moves the medical data corresponding to the movement condition from the high-speed storage 210 to the low-speed storage 220 based on the movement instruction of the control apparatus 200. In this case, the control apparatus 200 functions as the movement section 203. At this time, the medical data stored in the high-speed storage 210 is deleted from the high-speed storage 210 along with the movement to the low-speed storage 220.

On the other hand, in a case in which the deletion suppressing flag is “true”, the controller 100 of the medical information management apparatus 10B transmits, to the data storage system 20B, a movement suppressing request to suppress the movement of the medical data stored in the high-speed storage 210 to the low-speed storage 220. In this case, the controller 100 functions as the movement suppressing request section 102.

Upon receiving the movement suppressing request from the medical information management apparatus 10B, the control apparatus 200 of the data storage system 20B performs control to suppress movement of the medical data corresponding to the movement condition from the high-speed storage 210 to the low-speed storage 220. In this case, the control apparatus 200 functions as the movement suppressing section 204. Thus, even the medical data corresponding to the movement condition is not moved from the high-speed storage 210 to the low-speed storage 220. Therefore, since the medical data of which the deletion suppressing flag is set to “true” is not moved, the medical data is not deleted from the high-speed storage 210.

### 1-2. Second Modification Example of First Embodiment

In the second modification example, the high-speed storage 150 and the low-speed storage 160 are provided not on the cloud but in the same medical facility as the medical information management apparatus 10C. Note that in the second modification example, common constituent elements to the above-described first embodiment are denoted by the same reference signs, and redundant description is omitted.

### [Configuration Example of Medical Information Management System 1C]

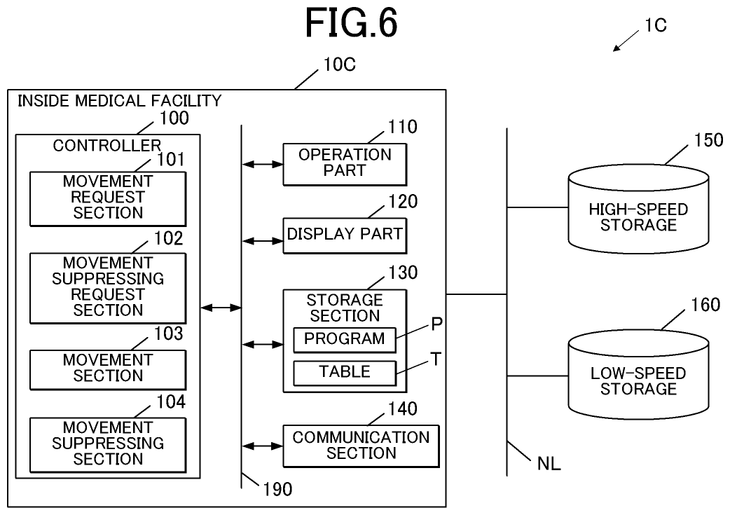

FIG. 6 is a diagram illustrating an example of a schematic configuration of the medical information management system 1C according to a second modification example of the first embodiment. The medical information management system 1C includes a medical information management apparatus 10C provided in a medical facility, a high-speed storage 150, and a low-speed storage 160.

The medical information management apparatus 10C includes a controller 100, an operation part 110, a display part 120, a storage section 130, and a communication section 140. The controller 100 functions as a movement request section 101, a movement suppressing request section 102, a movement section 103, and a movement suppressing section 104. The processor of the controller 100 executes a program P stored in the storage section 130 or the like to implement each function of the movement request section 101 or the like. Note that although the medical information management apparatus 10C is configured to include the operation part 110 and the display part 120 in FIG. 6, the medical information management apparatus 10C may be configured not to include the operation part 110 and the display part 120. For example, in each user terminal installed in the medical facility or the like, each doctor may display the examination list screen 120a or the like on a browser and input an instruction or the like to suppress deletion of medical data.

Each of the high-speed storage 150 and the low-speed storage 160 is connected to the medical information management apparatus 10C via the network NL. The network NL may be, for example, a LAN. LAN is an abbreviation for Local Area Network. The network NL may be a wired network or a wireless network.

### [Operation Example of Medical Information Management System 1C]

Next, a description will be given of a flow in a case in which processing such as movement of the medical data stored in the high-speed storage 150 to the low-speed storage 160 and suppression of the movement is executed according to the second modification example.

The controller 100 of the medical information management apparatus 10C acquires the medical data from the modality, and stores the acquired medical data in the storage section 130. After storing the medical data acquired from the modality in the storage section 130, the controller 100 transmits the medical data to the high-speed storage 150 via the network NL. The high-speed storage 150 stores the medical data transmitted from the medical information management apparatus 10C.

The controller 100 determines whether or not the medical data stored in the high-speed storage 150 corresponds to a movement condition set in advance. When the medical data stored in the high-speed storage 150 satisfies the movement condition, the controller 100 determines whether the deletion suppressing flag of the medical data is set to “false” or “true”. When the deletion suppressing flag is “false”, the controller 100 transmits, to the high-speed storage 150, a movement instruction based on a request to move the medical data from the high-speed storage 150 to the low-speed storage 160. In this case, the controller 100 functions as the movement request section 101 and the movement section 103.

Upon receiving the movement instruction from the medical information management apparatus 10C, the high-speed storage 150 moves the medical data corresponding to the movement condition to the low-speed storage 160. At this time, the medical data stored in the high-speed storage 150 is deleted from the high-speed storage 150 along with the movement to the low-speed storage 160.

On the other hand, when the deletion suppressing flag is “true”, the controller 100 executes control to suppress the movement of the medical data stored in the high-speed storage 150 to the low-speed storage 160. In this case, the controller 100 functions as a movement suppressing request section 102 and a movement suppressing section 104. Thus, even the medical data corresponding to the movement condition is not moved from the high-speed storage 150 to the low-speed storage 160. Therefore, since the medical data of which the deletion suppressing flag is set to “true” is not moved, the medical data is not deleted from the high-speed storage 150.

### 1-3. Third Modification Example of First Embodiment

In the third modification example, the high-speed storage 150 is provided in the medical facility, and the low-speed storage 220 is provided on a cloud. Note that in the third modification example, common constituent elements to the above-described first embodiment are denoted by the same reference signs, and redundant description is omitted.

### [Example of Configuration of Medical Information Management System 1D]

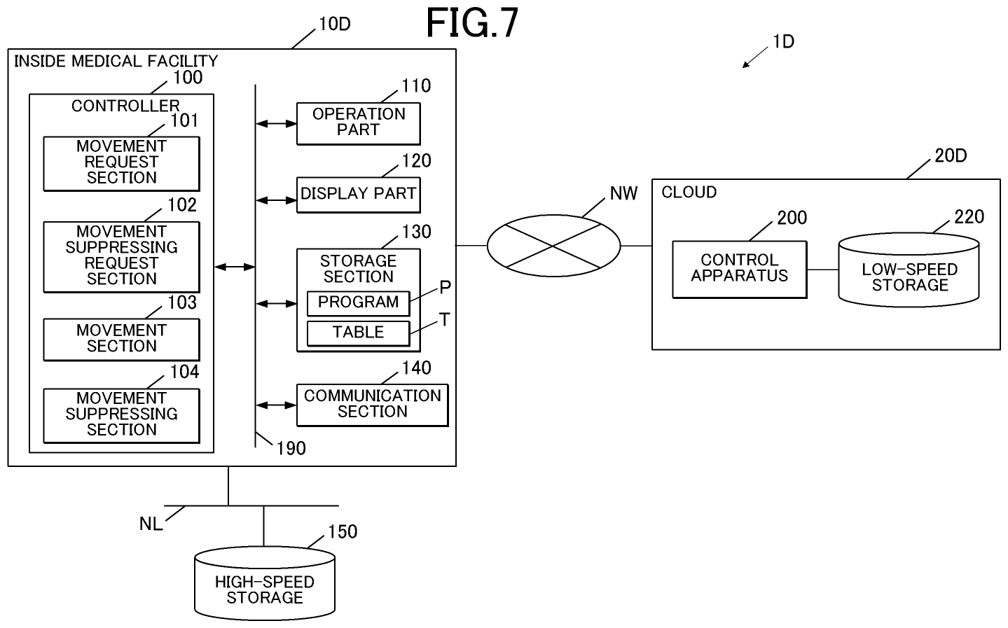

FIG. 7 is a diagram illustrating an example of a schematic configuration of the medical information management system 1D according to a third modification example of the first embodiment. The medical information management system 1D includes a medical information management apparatus 10D provided in the medical facility and a data storage system 20D provided on a cloud.

The medical information management apparatus 10D includes a controller 100, an operation part 110, a display part 120, a storage section 130, and a communication section 140. The controller 100 functions as a movement request section 101, a movement suppressing request section 102, a movement section 103, and a movement suppressing section 104. The processor of the controller 100 executes a program P stored in the storage section 130 or the like to implement each function of the movement request section 101 or the like. Note that although the medical information management apparatus 10D is configured to include the operation part 110 and the display part 120 in FIG. 7, the medical information management apparatus 10D may be configured not to include the operation part 110 and the display part 120. For example, in each user terminal installed in the medical facility or the like, each doctor may display the examination list screen 120a or the like on a browser and input an instruction or the like to suppress deletion of medical data.

A high-speed storage 150 is connected to the medical information management apparatus 10D via the network NL. The network NL may be, for example, a LAN. The data storage system 20D includes a control apparatus 200 and a low-speed storage 220.

### [Operation Example of Medical Information Management System 1D]

Next, a description will be given of a flow in a case in which processing such as movement of the medical data stored in the high-speed storage 150 to the low-speed storage 220 and suppression of the movement is executed according to the third modification example.

The controller 100 of the medical information management apparatus 10D acquires the medical data from the modality, and stores the acquired medical data in the storage section 130. After storing the medical data acquired from the modality in the storage section 130, the controller 100 transmits the medical data to the high-speed storage 150 via the network NL. The high-speed storage 150 stores therein the medical data transmitted from the medical information management apparatus 10D.

The controller 100 determines whether or not the medical data stored in the high-speed storage 150 corresponds to a movement condition set in advance. When the medical data stored in the high-speed storage 150 satisfies the movement condition, the controller 100 determines whether the deletion suppressing flag of the medical data is set to “false” or “true”. When the deletion suppressing flag is “false”, the controller 100 transmits, to the high-speed storage 150, a movement instruction based on a request to move the medical data corresponding to the movement condition from the high-speed storage 150 to the low-speed storage 220. In this case, the controller 100 functions as the movement request section 101 and the movement section 103.

Upon receiving the movement instruction from the medical information management apparatus 10D, the high-speed storage 150 transmits the medical data corresponding to the movement condition to the low-speed storage 220 in the data storage system 20D via the network NW. The low-speed storage 220 stores the medical data transmitted from the high-speed storage 150. At this time, the medical data stored in the high-speed storage 150 is deleted from the high-speed storage 150 along with the movement to the low-speed storage 220.

On the other hand, when the deletion suppressing flag is “true”, the controller 100 of the medical information management apparatus 10D executes control to suppress the movement of the medical data stored in the high-speed storage 150 to the low-speed storage 220. In this case, the controller 100 functions as a movement suppressing request section 102 and a movement suppressing section 104. Thus, even the medical data corresponding to the movement condition is not moved from the high-speed storage 150 to the low-speed storage 220. Therefore, since the medical data of which the deletion suppressing flag is set to “true” is not moved, the medical data is not deleted from the high-speed storage 150.

### 2. Second Embodiment

The second embodiment is different from the first embodiment in that the medical data stored in the high-speed storage 210 is not moved but copied to the low-speed storage 220. Note that in the second embodiment, common constituent elements to the above-described first embodiment are denoted by the same reference signs, and redundant description is omitted.

### [Schematic Configuration Example of Medical Information Management System 1E]

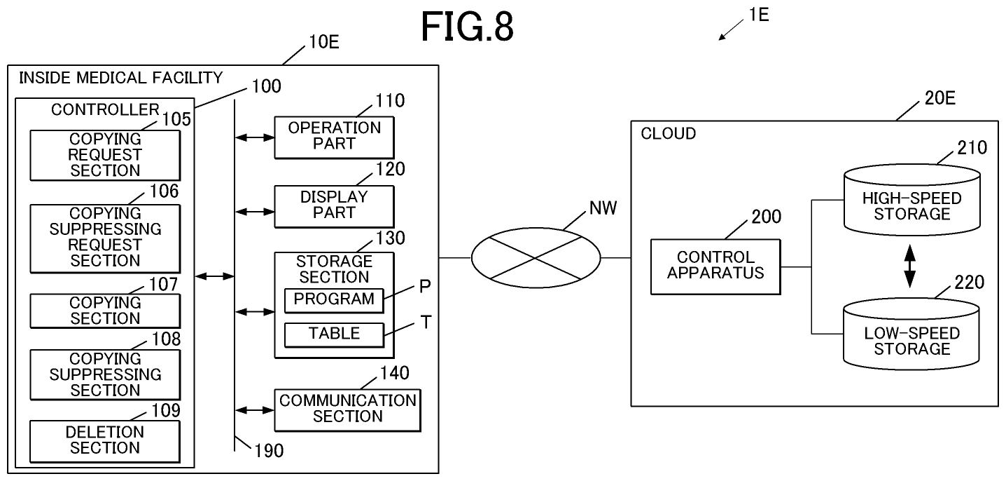

FIG. 8 is a diagram illustrating an example of a schematic configuration of the medical information management system 1E according to the second embodiment. The medical information management system 1E includes a medical information management apparatus 10E provided in the medical facility and a data storage system 20E provided on a cloud. The medical information management apparatus 10E and the data storage system 20E are communicably connected to each other via a network NW.

The medical information management apparatus 10E includes a controller 100, an operation part 110, a display part 120, a storage section 130, and a communication section 140. The controller 100 functions as a copying request section 105, a copying suppressing request section 106, a copying section 107, a copying suppressing section 108, and a deletion section 109. The processor of the controller 100 executes the program P stored in the storage section 130 or the like to implement each function of the copying request section 105 or the like. Note that although the medical information management apparatus 10E is configured to include the operation part 110 and the display part 120 in FIG. 8, the medical information management apparatus 10E may be configured not to include the operation part 110 and the display part 120. For example, in each user terminal installed in the medical facility or the like, each doctor may display the examination list screen 120a or the like on a browser and input an instruction or the like to suppress copying of medical data.

The copying request section 105 makes a request to store the medical data in the low-speed storage 220 by making a request to copy the medical data stored in the high-speed storage 210 to the low-speed storage 220. Copying means storing medical data stored in the high-speed storage 210 into the low-speed storage 220, but not deleting the medical data stored in the high-speed storage 210. The copying request by the copying request section includes setting of copying conditions in advance. The copying condition may be set for all or a part of the medical data stored in the high-speed storage 210.

The copying suppressing request section 106 requests control to suppress copying of the medical data for which suppressing of copying has been instructed by an instruction section such as the examination list screen 120a. Note that the examination list screen 120a in FIG. 2 relates to the movement of the medical data, but is also applicable to the medical data transfer in the second embodiment. The suppressing of copying can be set by changing the deletion suppressing flag to be enabled on an examination basis or a patient basis with the instruction section such as the examination list screen 120a. The copying suppressing request section 106 may determine whether the deletion suppressing flag of the patient is valid.

After the medical data is stored in the high-speed storage 210, the copying section 107 performs control to copy the medical data stored in the high-speed storage 210 to the low-speed storage 220 on the basis of a copying request of the copying request section 105. In this case, the copying section 107 performs automatic copying control based on preset copying conditions. The copying suppressing section 108 performs control to suppress copying of the medical data stored in the high-speed storage 210 to the low-speed storage 220, based on the request of the copying suppressing request section 106. The deletion section 109 performs control to automatically delete the medical data from the high-speed storage 210 based on a deletion condition for deleting the medical data stored in the high-speed storage 210. When a deletion condition is satisfied for the copied medical data, for example, when a certain storage period has elapsed, the deletion section 109 deletes the medical data from the high-speed storage 210.

The data storage system 20E includes a control apparatus 200, a high-speed storage 210, and a low-speed storage 220. Based on each instruction transmitted from the medical information management apparatus 10E, the data storage system 20E copies the medical data stored in the high-speed storage 210 to the low-speed storage 220, or suppresses copying of the medical data stored in the high-speed storage 210 to the low-speed storage 220.

### [Operation Example of Medical Information Management System 1E]

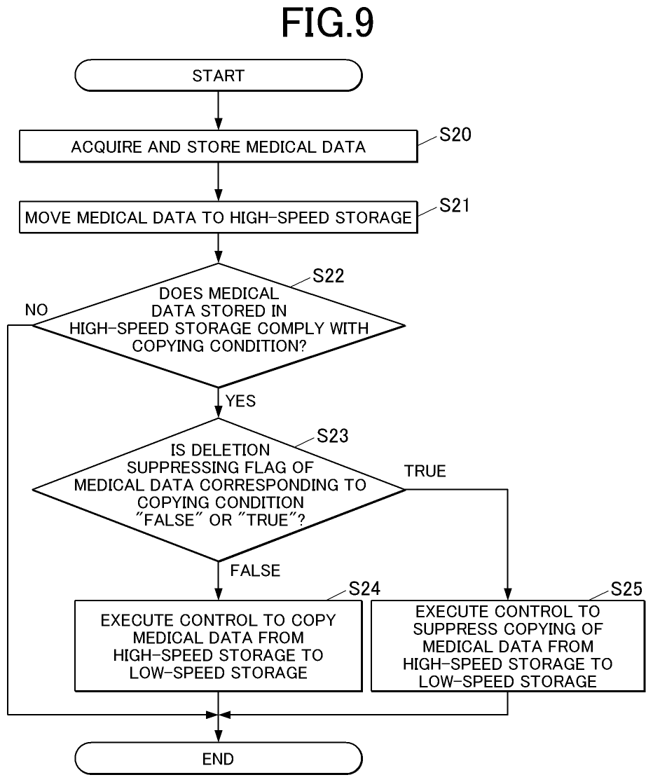

Next, a flow of processing such as copying of medical data stored in the high-speed storage 210 according to the present embodiment to the low-speed storage 220 and suppressing of the copying will be described. FIG. 9 is a flowchart illustrating an example of an operation of the medical information management system 1E according to the second embodiment in a case in which control such as copying of medical data stored in the high-speed storage 210 to the low-speed storage 220 and suppressing of the copying is executed.

The controller 100 of the medical information management apparatus 10E acquires the medical data from the modality, and stores the acquired medical data in the storage section 130 (step S20).

After storing the medical data acquired from the modality in the storage section 130, the controller 100 stores the medical data in the high-speed storage 210 of the data storage system 20E (step S21). Specifically, the controller 100 transmits the acquired medical data to the high-speed storage 210 via the network NW. The high-speed storage 210 stores therein the medical data transmitted from the medical information management apparatus 10E.

The controller 100 determines whether the medical data stored in the high-speed storage 210 satisfies a copying condition set in advance (step S22). The copying conditions are the same as the movement conditions described in the first embodiment, and therefore, a detailed description is omitted. In a case where the controller 100 determines that the medical data stored in the high-speed storage 210 satisfies the copying condition, the process proceeds to step S23. On the other hand, when determining that the medical data stored in the high-speed storage 210 does not correspond to the copying condition, the controller 100 ends the series of processing without copying the medical data from the high-speed storage 210.

The controller 100 determines whether the deletion suppressing flag of the medical data corresponding to the copying condition is set to “false” or “true” (step S23). The controller 100 can determine whether the deletion suppressing flag is “false” or “true” using the table T described in the first embodiment. When determining that the deletion suppressing flag is set to “false” for the medical data corresponding to the copying condition, the controller 100 proceeds to step S24.

In this case, the controller 100 sends, to the data storage system 20E, a copying instruction based on the request to copy the medical data corresponding to the copying conditions from the high-speed storage 210 to the low-speed storage 220. That is, the controller 100 executes control to copy the medical data corresponding to the copying condition from the high-speed storage 210 to the low-speed storage 220 (step S24). In this case, the controller 100 functions as a copying request section 105 and a copying section 107.

Upon receiving the copying instruction from the medical information management apparatus 10E, the high-speed storage 210 of the data storage system 20E copies the medical data corresponding to the copying condition to the low-speed storage 220. At this time, the controller 100 does not delete the copied medical data stored in the high-speed storage 210. After the copying, when the controller 100 of the medical information management apparatus 10E determines that the copied medical data satisfies a predetermined deletion condition, the controller 100 deletes the copied medical information from the high-speed storage 210. In this case, the controller 100 functions as the deletion section 109.

On the other hand, if the controller 100 determines in step S23 that the deletion suppressing flag for the medical data corresponding to the copying condition is set to “true”, the controller 100 proceeds to step S25.

The controller 100 executes control to suppress copying of the medical data corresponding to the copying condition from the high-speed storage 210 to the low-speed storage 220. In this case, the controller 100 functions as a copying suppressing request section 106 and a copying suppressing section 108. Thus, even the medical data corresponding to the copying condition is not copied from the high-speed storage 210 to the low-speed storage 220. Therefore, since the medical data of which the deletion suppressing flag is set to “true” is not copied, the medical data is not deleted from the high-speed storage 210.

According to the second embodiment, by enabling the deletion suppressing flag of one patient or the like on the examination list screen 120a or the like, it is possible to designate in advance the medical data for which the automatic copying of the medical data corresponding to the copying condition from the high-speed storage 210 to the low-speed storage 220 is suppressed. Thus, even when the medical data corresponds to the copying condition, the medical data can be left in the high-speed storage 210 by enabling the deletion suppressing flag, and undesired copying to the low-speed storage 220 can be suppressed. As a result, for example, even medical data that is infrequently referred to can be referred to from the high-speed storage 210, thus reducing the standby time until it can be referred to. Regarding other effects, the same effects as those of the first embodiment can be achieved.

### 2-1. First Modification Example of Second Embodiment

In the first modification example, the data storage system 20F provided on the cloud executes the copying of the medical data stored in the high-speed storage 210 to the low-speed storage 220 and the suppressing of the copying. Note that in the first modification example, common constituent elements to the above-described second embodiment are denoted by the same reference signs, and redundant description is omitted.

### [Configuration Example of Medical Information Management System 1F]

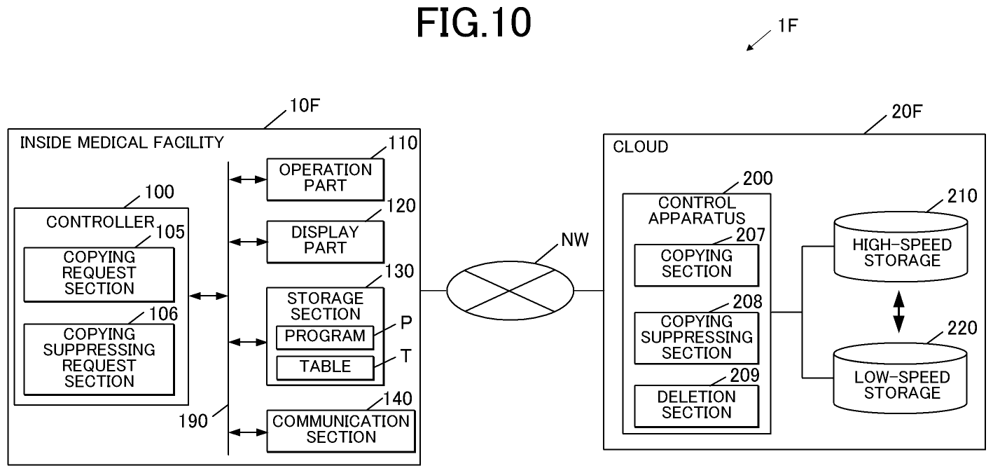

FIG. 10 shows an example of the schematic configuration of a medical information management system 1F according to the first modification example of the second embodiment. The medical information management system 1F includes a medical information management apparatus 10F provided in the medical facility and a data storage system 20F provided on a cloud.

The medical information management apparatus 10F includes a controller 100, an operation part 110, a display part 120, a storage section 130, and a communication section 140. The controller 100 functions as a copying request section 105 and a copying suppressing request section 106. The processor of the controller 100 executes the program P stored in the storage section 130 or the like to implement each function of the copying request section 105 or the like. Note that although the medical information management apparatus 10F is configured to include the operation part 110 and the display part 120 in FIG. 10, the medical information management apparatus 10F may be configured not to include the operation part 110 and the display part 120. For example, in each user terminal installed in the medical facility or the like, each doctor may display the examination list screen 120a or the like on a browser and input an instruction or the like to suppress copying of medical data.

The data storage system 20F includes a control apparatus 200, a high-speed storage 210, and a low-speed storage 220. The control apparatus 200 functions as a copying section 207, a copying suppressing section 208, and a deletion section 209. The processor of the control apparatus 200 executes a program stored in a memory or the like (not illustrated) to realize each function of the copying section 207 and the like.

### [Operation Example of Medical Information Management System 1F]

Next, a flow of processing in a case in which the medical data stored in the high-speed storage 210 according to the first modification example is copied to the low-speed storage 220 and the copying is suppressed will be described.

The controller 100 of the medical information management apparatus 10F acquires the medical data from the modality, and stores the acquired medical data in the storage section 130. After storing the medical data acquired from the modality in the storage section 130, the controller 100 transmits the medical data to the high-speed storage 210 of the data storage system 20F via the network NW. The high-speed storage 210 stores therein the medical data transmitted from the medical information management apparatus 10F.

The controller 100 determines whether or not the medical data stored in the high-speed storage 210 corresponds to a copying condition set in advance. When the medical data stored in the high-speed storage 210 satisfies the copying condition, the controller 100 determines whether the deletion suppressing flag of the medical data is set to “false” or “true”. When the deletion suppressing flag is “false”, the controller 100 transmits, to the data storage system 20F, a copying request for copying the medical data from the high-speed storage 210 to the low-speed storage 220. In this case, the controller 100 functions as the copying request section 105.

Upon receiving the copying request from the medical information management apparatus 10F, the control apparatus 200 of the data storage system 20F outputs, to the high-speed storage 210, a copying instruction to copy the medical data corresponding to the copying condition from the high-speed storage 210 to the low-speed storage 220. In this case, the control apparatus 200 functions as the copying section 207. The high-speed storage 210 copies the medical data corresponding to the copying condition from the high-speed storage 210 to the low-speed storage 220 based on the copying instruction of the control apparatus 200. During the copying, the control apparatus 200 does not delete the medical data stored in the high-speed storage 210. After the copying, when determining that the copied medical data satisfies a certain deletion condition, the control apparatus 200 deletes the copied medical data from the high-speed storage 210. In this case, the control apparatus 200 functions as the deletion section 209.

On the other hand, when the deletion suppressing flag is “true”, the controller 100 of the medical information management apparatus 10F transmits, to the data storage system 20F, a copying suppressing request to suppress the copying of the medical data stored in the high-speed storage 210 to the low-speed storage 220. In this case, the controller 100 functions as the copying suppressing request section 106.

Upon receiving the copying suppressing request from the medical information management apparatus 10F, the control apparatus 200 of the data storage system 20F performs control to suppress copying of the medical data corresponding to the copying condition from the high-speed storage 210 to the low-speed storage 220. In this case, the control apparatus 200 functions as the copying suppressing section 208. Thus, even the medical data corresponding to the copying condition is not copied from the high-speed storage 210 to the low-speed storage 220. Therefore, since the medical data of which the deletion suppressing flag is set to “true” is not copied, the medical data is not deleted from the high-speed storage 210.

### 2-2. Second Modification Example of Second Embodiment

In the second modification example, the high-speed storage 150 and the low-speed storage 160 are provided not on the cloud but in the same medical facility as the medical information management apparatus 10G. Note that in the second modification example, common constituent elements to the above-described second embodiment are denoted by the same reference signs, and redundant description is omitted.

### [Example of Configuration of Medical Information Management System 1G]

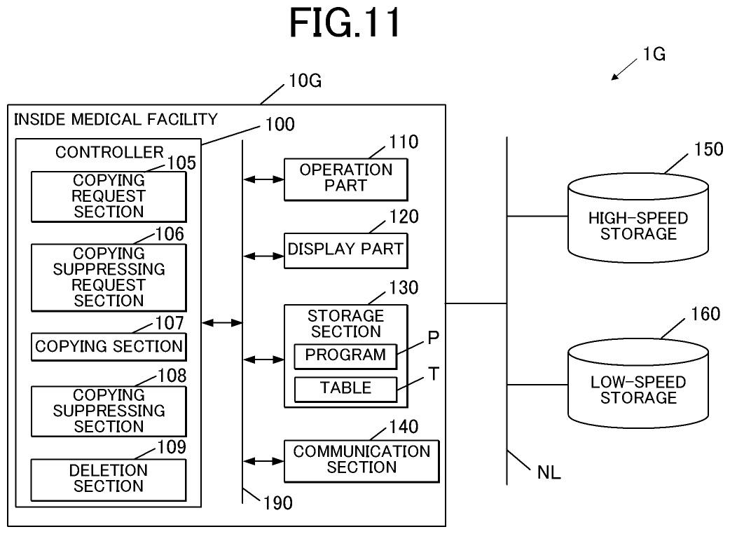

FIG. 11 is a diagram illustrating an example of a schematic configuration of the medical information management system 1G according to a second modification example of the second embodiment. The medical information management system 1G includes a medical information management apparatus 10G, a high-speed storage 150, and a low-speed storage 160. The medical information management apparatus 10G, the high-speed storage 150, and the low-speed storage 160 are installed in a medical facility.

The medical information management apparatus 10G includes a controller 100, an operation part 110, a display part 120, a storage section 130, and a communication section 140. The controller 100 functions as a copying request section 105, a copying suppressing request section 106, a copying section 107, a copying suppressing section 108, and a deletion section 109. The processor of the controller 100 executes the program P stored in the storage section 130 or the like to implement each function of the copying request section 105 or the like. Note that although the medical information management apparatus 10G is configured to include the operation part 110 and the display part 120 in FIG. 11, the medical information management apparatus 10G may be configured not to include the operation part 110 and the display part 120. For example, in each user terminal installed in the medical facility or the like, each doctor may display the examination list screen 120a or the like on a browser and input an instruction or the like to suppress copying of medical data.

Each of the high-speed storage 150 and the low-speed storage 160 is connected to the medical information management apparatus 10G via the network NL. The network NL may be, for example, a LAN. LAN is an abbreviation for Local Area Network. The communication method may be wired or wireless.

### [Operation Example of Medical Information Management System 1G]

Next, a description will be given of a flow of processing in a case of executing copying of the medical data stored in the high-speed storage 210 to the low-speed storage 220 and suppressing the copying, according to the second modification example.

The controller 100 of the medical information management apparatus 10G acquires the medical data from the modality, and stores the acquired medical data in the storage section 130. After storing the medical data acquired from the modality in the storage section 130, the controller 100 transmits the medical data to the high-speed storage 150 via the network NL. The high-speed storage 150 stores therein the medical data transmitted from the medical information management apparatus 10G.

The controller 100 determines whether or not the medical data stored in the high-speed storage 150 corresponds to a copying condition set in advance.

When the medical data stored in the high-speed storage 150 satisfies the copying condition, the controller 100 determines whether the deletion suppressing flag of the medical data is set to “false” or “true”. When the deletion suppressing flag is “false”, the controller 100 transmits, to the high-speed storage 150, a copying instruction based on a request to copy the medical data from the high-speed storage 210 to the low-speed storage 220. In this case, the controller 100 functions as a copying request section 105 and a copying section 107.

Upon receiving the copying instruction from the medical information management apparatus 10G, the high-speed storage 150 copies the medical data corresponding to the copying condition to the low-speed storage 160. At this time, the controller 100 does not delete the medical data stored in the high-speed storage 210. After the copying, when the controller 100 determines that the copied medical data satisfies a deletion condition set in advance, the controller 100 deletes the copied medical data from the high-speed storage 210.

On the other hand, when the deletion suppressing flag is “true”, the controller 100 executes control to suppress the copying of the medical data stored in the high-speed storage 150 to the low-speed storage 160. In this case, the controller 100 functions as a copying suppressing request section 106 and a copying suppressing section 108. Thus, even the medical data corresponding to the copying condition is not copied from the high-speed storage 150 to the low-speed storage 160. Therefore, since the medical data of which the deletion suppressing flag is set to “true” is not copied, the medical data is not deleted from the high-speed storage 210.

### 2-3. Third Modification Example of Second Embodiment

In the third modification example, the high-speed storage 150 is provided in the medical facility, and the low-speed storage 220 is provided on a cloud. Note that in the third modification example, common constituent elements to the above-described second embodiment are denoted by the same reference signs, and redundant description is omitted.

### [Example of Configuration of Medical Information Management System 1H]

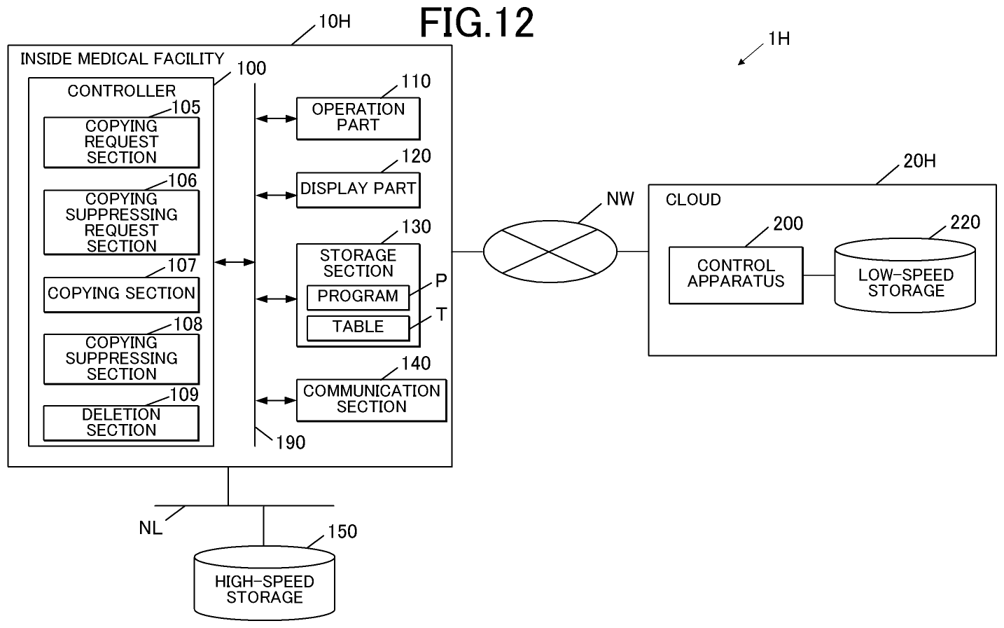

FIG. 12 is a diagram illustrating an example of a schematic configuration of the medical information management system 1H according to a third modification example of the second embodiment. The medical information management system 1H includes a medical information management apparatus 10H provided in a medical facility and a data storage system 20H provided on a cloud. The medical information management apparatus 10H and the data storage system 20H are communicably connected to each other via a network NW.

The medical information management apparatus 10H includes a controller 100, an operation part 110, a display part 120, a storage section 130, and a communication section 140. The controller 100 functions as a copying request section 105, a copying suppressing request section 106, a copying section 107, a copying suppressing section 108, and a deletion section 109. The processor of the controller 100 executes the program P stored in the storage section 130 or the like to implement each function of the copying request section 105 or the like. Note that although the medical information management apparatus 10H is configured to include the operation part 110 and the display part 120 in FIG. 12, the medical information management apparatus 10H may be configured not to include the operation part 110 and the display part 120. For example, in each user terminal installed in the medical facility or the like, each doctor may display the examination list screen 120a or the like on a browser and input an instruction or the like to suppress copying of medical data.

A high-speed storage 150 is connected to the medical information management apparatus 10H via the network NL. The network NL may be, for example, a LAN. The data storage system 20H includes a control apparatus 200 and a low-speed storage 220.

### [Operation Example of Medical Information Management System 1H]

Next, a description will be given of a flow of processing in a case of executing copying of the medical data stored in the high-speed storage 210 to the low-speed storage 220 and suppressing the copying, according to the third modification example.

The controller 100 of the medical information management apparatus 10H acquires the medical data from the modality, and stores the acquired medical data in the storage section 130. After storing the medical data acquired from the modality in the storage section 130, the controller 100 transmits the medical data to the high-speed storage 150 in the medical facility via the network NL. The high-speed storage 150 stores therein the medical data transmitted from the medical information management apparatus 10E.

The controller 100 determines whether or not the medical data stored in the high-speed storage 150 corresponds to a copying condition set in advance. When the medical data stored in the high-speed storage 150 satisfies the copying condition, the controller 100 determines whether the deletion suppressing flag of the medical data is set to “false” or “true”. When the deletion suppressing flag is “false”, the controller 100 transmits, to the high-speed storage 150, a copying instruction including a request to copy the medical data satisfying the copying conditions from the high-speed storage 150 to the low-speed storage 220. In this case, the controller 100 functions as a copying request section 105 and a copying section 107.

Upon receiving the copying instruction from the medical information management apparatus 10H, the high-speed storage 150 transmits the medical data corresponding to the copying condition to the low-speed storage 220 of the data storage system 20H via the network NW. The low-speed storage 220 stores the medical data transmitted from the high-speed storage 150. At this time, the controller 100 of the medical information management apparatus 10H does not delete the medical data stored in the high-speed storage 210. After the copying, when the controller 100 determines that the copied medical data satisfies a deletion condition set in advance, the controller 100 deletes the copied medical data from the high-speed storage 210.

On the other hand, when the deletion suppressing flag is “true”, the controller 100 executes control to suppress copying of the medical data stored in the high-speed storage 150 to the low-speed storage 220. In this case, the controller 100 functions as a copying suppressing request section 106 and a copying suppressing section 108. Thus, even the medical data corresponding to the copying condition is not copied from the high-speed storage 150 to the low-speed storage 220. Therefore, since the medical data of which the deletion suppressing flag is set to “true” is not copied, the medical data is not deleted from the high-speed storage 210.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

Although embodiments of the present invention have been described and shown in detail, the disclosed embodiments are made for purposes of illustration and example only and not limitation. The scope of the present invention should be interpreted by terms of the appended claims.

The entire disclosure of Japanese Patent Application No. 2024-041871, filed on Mar. 18, 2024, including description, claims, drawings and abstract is incorporated herein by reference.

What is claimed is:

1. A medical information management apparatus comprising:
a hardware processor,
wherein,
the hardware processor requests that medical data stored in a first storage apparatus having a predetermined access speed be stored in a second storage device having a lower access speed than the first storage device,
the hardware processor instructs suppressing deletion of the medical data stored in the first storage device, and
the hardware processor requests control to suppress the deletion of the medical data for which the suppressing of the deletion is instructed.

2. The medical information management apparatus according to claim 1, wherein,
the hardware processor requests to store by requesting to move the medical data stored in the first storage device to the second storage device,
the hardware processor suppresses the deletion by suppressing the movement of the medical data stored in the first storage device, and
the hardware processor requests control to suppress the deletion by suppressing the movement of the medical data for which the suppressing of the movement is instructed.

3. The medical information management apparatus according to claim 1, wherein,
the hardware processor requests to store by requesting to copy the medical data stored in the first storage device to the second storage device,
the hardware processor performs control to automatically delete the medical data from the first storage device based on a deletion condition to delete the medical data stored in the first storage device,
the hardware processor instructs suppressing the deletion of the medical data stored in the first storage device, and
the hardware processor requests the control to suppress the deletion of the medical data for which the suppressing of the deletion is instructed.

4. The medical information management apparatus according to claim 1, wherein the hardware processor instructs suppressing the deletion of the medical data of one patient by one instruction with respect to the medical data stored in the first storage device.

5. The medical information management apparatus according to claim 1, wherein the hardware processor instructs suppressing the deletion of the medical data of one examination by one instruction with respect to the medical data stored in the first storage device.

6. The medical information management apparatus according to claim 1, wherein the hardware processor requests control to suppress the deletion of the medical data determined to be deleted based on a predetermined deletion condition among the medical data stored in the first storage device.

7. A medical information management system comprising:
a hardware processor,
wherein,
the hardware processor requests that medical data stored in a first storage apparatus having a predetermined access speed be stored in a second storage device having a lower access speed than the first storage device,
the hardware processor instructs suppressing deletion of the medical data stored in the first storage device, and
the hardware processor requests control to suppress the deletion of the medical data for which the suppressing of the deletion is instructed.

8. A medical information management method comprising:
requesting that medical data stored in a first storage apparatus having a predetermined access speed be stored in a second storage device having a lower access speed than the first storage device;
instructing suppressing deletion of the medical data stored in the first storage device; and
requesting control to suppress the deletion of the medical data for which the suppressing of the deletion is instructed.

9. A non-transitory computer-readable storage medium comprising a program for causing a computer to perform:
requesting that medical data stored in a first storage apparatus having a predetermined access speed be stored in a second storage device having a lower access speed than the first storage device;
instructing suppressing deletion of the medical data stored in the first storage device; and
requesting control to suppress the deletion of the medical data for which the suppressing of the deletion is instructed.
